(19) United States
(12) Patent Application Publication
(10) Pub. No.: US 2025/0329784 A1
(43) Pub. Date: Oct. 23, 2025

(54) ELECTROLYTE SOLUTION FOR RECHARGEABLE LITHIUM BATTERY AND RECHARGEABLE LITHIUM BATTERY INCLUDING THE SAME

(71) Applicant: SAMSUNG SDI CO., LTD., Yongin-si (KR)

(72) Inventors: Yeji YANG, Yongin-si (KR); Sanghoon KIM, Yongin-si (KR); Hyunbong CHOI, Yongin-si (KR); Sangwoo PARK, Yongin-si (KR); Sohee KIM, Yongin-si (KR); Hongryeol PARK, Yongin-si (KR); Dasol JUN, Yongin-si (KR); Youngkyeong SHIN, Yongin-si (KR); Woojung JI, Yongin-si (KR); Sundae KIM, Yongin-si (KR); Euisoo JEONG, Yongin-si (KR)

(73) Assignee: SAMSUNG SDI CO., LTD., Yongin-si (KR)

(21) Appl. No.: 18/951,345

(22) Filed: Nov. 18, 2024

(30) Foreign Application Priority Data
Apr. 18, 2024 (KR) 10-2024-0051944

(51) Int. Cl. H01M 10/0567; H01M 10/052; H01M 10/0568; H01M 10/0569

(52) U.S. Cl. CPC H01M 10/0567; H01M 10/052; H01M 10/0568; H01M 10/0569; H01M 2300/0028

(57) ABSTRACT

An electrolyte solution for a rechargeable lithium battery and a rechargeable lithium battery including the same, wherein the electrolyte solution includes a non-aqueous organic solvent, a lithium salt, and an additive.

### CROSS-REFERENCE TO RELATED APPLICATIONS

This U.S. non-provisional patent application claims priority under 35 U.S.C. § 119 to Korean Patent Application No. 10-2024-0051944, filed on Apr. 18, 2024, the entire contents of which are hereby incorporated by reference.

### BACKGROUND

Examples of the present disclosure relates to an electrolyte solution for a rechargeable lithium battery and a rechargeable lithium battery including the electrolyte solution.

The increased use of battery-powered electronics such as, e.g., mobile phones, laptop computers, electric vehicles, and the like, has increased demand for rechargeable batteries provided with high energy density and high capacity.

Rechargeable lithium batteries typically include a positive electrode and a negative electrode, each of the positive electrode and the negative electrode including an active material that allows intercalation and deintercalation of lithium ions, and an electrolyte solution. The rechargeable lithium battery generates electrical energy from redox reactions that take place as lithium ions are intercalated into or deintercalated from the positive electrode and the negative electrode.

In an example, in which a lithium salt is dissolved in a non-aqueous organic solvent and constitutes an electrolyte of the rechargeable lithium batteries. The rechargeable lithium batteries exhibit characteristics thereof through complex reactions between the positive electrode and the electrolyte, and between the negative electrode and the electrolyte. Thus, the use of an appropriate electrolyte is a compelling variable in improving performance of the rechargeable lithium batteries.

### SUMMARY

Examples of present disclosure include an electrolyte solution for a rechargeable lithium battery, the electrolyte solution having improved lifetime characteristics and stability at high temperatures.

Examples of the present disclosure also include a rechargeable lithium battery including the electrolyte solution.

An example embodiment of the present disclosure includes an electrolyte solution for a rechargeable lithium battery, the electrolyte solution including at least a non-aqueous organic solvent, a lithium salt, and an additive represented by Formula 1 below.

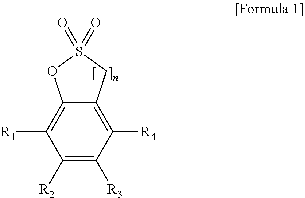

In Formula 1 above,
R₁ to R₄ are each independently a hydrogen atom, a halogen atom, or a substituted or unsubstituted C₁ to C₁₀ alkyl group, and
n is an integer ranging from 1 to 5.

In an example embodiment of the present disclosure, a rechargeable lithium battery includes a positive electrode including a positive electrode active material, a negative electrode including a negative electrode active material, and an electrolyte solution. The electrolyte solution includes a non-aqueous organic solvent, a lithium salt, and an additive represented by Formula 1 described above.

In an example embodiment of the present disclosure, an electrolyte additive compound for a rechargeable lithium battery is represented by Formula 1-1 below:

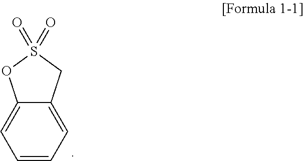 [SMILES: O=S1(=O)Cc2ccccc2O1]

### BRIEF DESCRIPTION OF THE FIGURES

The accompanying drawings are included to provide a further understanding of the present disclosure, and are incorporated in and constitute a part of this specification. The drawings illustrate example embodiments of the present disclosure and, together with the description, explain principles of the present disclosure. In the drawings:

FIG. 1 is a simplified conceptual view showing a rechargeable lithium battery according to example embodiments of the present disclosure; and

FIGS. 2 to 5 are cross-sectional views schematically showing a rechargeable lithium battery according to an example embodiment.

### DETAILED DESCRIPTION

In order to sufficiently understand the configuration and effects of the present disclosure, example embodiments of the present disclosure will be described with reference to the accompanying drawings. It should be noted, however, that the present disclosure is not limited to the following example embodiments, and may be implemented in various forms and variously modified. The example embodiments herein are provided so that the present disclosure is thorough and complete, and fully conveys the scope of the present disclosure to those skilled in the art.

Herein, it will be understood that when a component is referred to as being on another component, the component may be directly on another component, or an intervening third component may be present. In addition, in the drawings, thicknesses of components are exaggerated for effectively describing technical contents. Like reference numerals refer to like elements throughout.

Unless otherwise specified herein, the expression of singular form may include the expression of plural form. In addition, unless otherwise specified, the phrase “A or B” may indicate “A but not B,” “B but not A,” or “A and B.” The terms “comprises” and/or “comprising” used herein do not exclude the presence or addition of one or more other components.

As used herein, the term “combination thereof” may refer to a mixture, a stack, a composite, a copolymer, an alloy, a blend, or a reaction product.

Herein, unless otherwise defined, “substitution” indicates that at least one hydrogen in a substituent or compound is substituted with deuterium, a halogen group, a hydroxyl group, an amino group, a C1 to C30 amine group, a nitro group, a C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 fluoroalkyl group, a cyano group, or a combination thereof.

Specifically, the “substitution” may indicate that at least one hydrogen in a substituent or compound is substituted with deuterium, a halogen group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C10 fluoroalkyl group, or a cyano group. For example, the “substitution” may indicate that at least one hydrogen in a substituent or compound is substituted with deuterium, a halogen group, a C1 to C20 alkyl group, a C6 to C30 aryl group, a C1 to C10 fluoroalkyl group, or a cyano group. In addition, the “substitution” may indicate that at least one hydrogen in a substituent or compound is substituted with deuterium, a halogen group, a C1 to C5 alkyl group, a C6 to C18 aryl group, a C1 to C5 fluoroalkyl group, or a cyano group. For example, the “substitution” may indicate that at least one hydrogen in a substituent or compound is substituted with deuterium, a cyano group, a halogen group, a methyl group, an ethyl group, a propyl group, a butyl group, a phenyl group, a biphenyl group, a terphenyl group, a trifluoromethyl group, or a naphthyl group.

When the terms “about” or “substantially” are used in this specification in connection with a numerical value, it is intended that the associated numerical value include a tolerance of ±10% around the stated numerical value. When ranges are specified, the range includes all values therebetween such as increments of 0.1%.

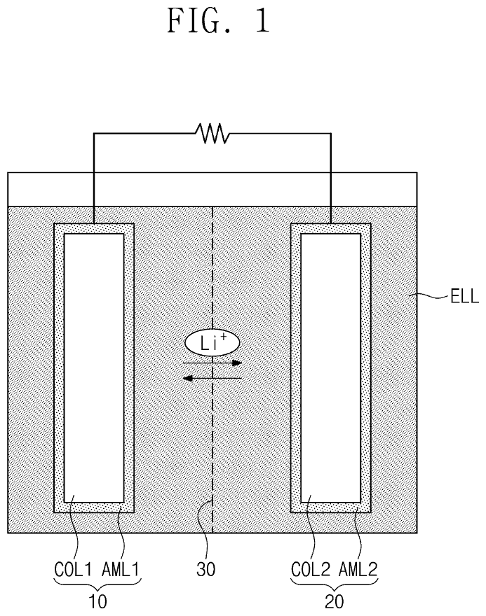

FIG. 1 is a cross-sectional view of a rechargeable lithium battery according to example embodiments of the present disclosure. Referring to FIG. 1, the rechargeable lithium battery may include a positive electrode 10, a negative electrode 20, a separator 30, and an electrolyte solution ELL.

The positive electrode 10 and the negative electrode 20 may be spaced apart from each other by the separator 30. The separator 30 may be between the positive electrode 10 and the negative electrode 20. The positive electrode 10, the negative electrode 20 and the separator 30 may be in contact with the electrolyte solution ELL. The positive electrode 10, the negative electrode 20 and the separator 30 may be impregnated in the electrolyte solution ELL.

The electrolyte solution ELL may constitute a medium for transferring lithium ions between the positive electrode 10 and the negative electrode 20. In the electrolyte solution ELL, the lithium ions may move through the separator 30 toward the positive electrode 10 or the negative electrode 20.

### Positive Electrode 10

The positive electrode 10 for a rechargeable lithium battery may include a current collector COL1 and a positive electrode active material layer AML1 on the current collector. The positive electrode active material layer AML1 may include a positive electrode active material, and may further include a binder and/or a conductive material (e.g., an electrically conductive material).

For example, the positive electrode 10 may further include an additive that can constitute a sacrificial positive electrode.

An amount of the positive electrode active material may be about 90 wt % to about 99.5 wt % based on 100 wt % of the positive electrode active material layer AML1. Amounts of the binder and the conductive material may be about 0.5 wt % to about 5 wt %, respectively, based on 100 wt % of the positive electrode active material layer AML1.

The binder is configured to sufficiently attach the positive electrode active material particles to each other and also to sufficiently attach the positive electrode active material to the current collector COLL. Examples of the binder may include at least one of polyvinyl alcohol, carboxymethyl cellulose, hydroxypropyl cellulose, diacetyl cellulose, polyvinylchloride, carboxylated polyvinylchloride, polyvinylfluoride, a polymer including ethylene oxide, polyvinylpyrrolidone, polyurethane, polytetrafluoroethylene, polyvinylidene fluoride, polyethylene, polypropylene, a styrene-butadiene rubber, a (meth)acrylated styrene-butadiene rubber, an epoxy resin, a (meth)acrylic resin, a polyester resin, nylon, and the like, as non-limiting examples.

The conductive material may impart conductivity (e.g., electrical conductivity) to the electrode. Any material that does not cause chemical change (e.g., that does not cause an undesirable chemical change in the rechargeable lithium battery) and that conducts electrons can be used in the battery. Examples of the conductive material may include at least one of a carbon-based material such as natural graphite, artificial graphite, carbon black, acetylene black, ketjen black, a carbon fiber, a carbon nanofiber, and carbon nanotube; a metal-based material containing at least one of copper, nickel, aluminum, silver, and the like, in a form of a metal powder or a metal fiber; a conductive polymer such as a polyphenylene derivative; or a mixture thereof.

Al may be used as the current collector COL1, but is not limited thereto.

### Positive Electrode Active Material

The positive electrode active material may include a compound (lithiated intercalation compound) that is capable of intercalating and deintercalating lithium. For example, at least one of a composite oxide of lithium and a metal including at least one of cobalt, manganese, nickel, and combinations thereof may be used.

The composite oxide may be or include a lithium transition metal composite oxide. Examples of the composite oxide may include at least one of lithium nickel-based oxide, lithium cobalt-based oxide, lithium manganese-based oxide, lithium iron phosphate-based compound, cobalt-free nickel-manganese-based oxide, or a combination thereof.

As an example, the following compounds represented by any one or more of the following Chemical Formulas may be used. LiₐA_{1-b}X_{b}O_{2-c}D_{c} (0.90≤a≤1.8, 0≤b≤0.5, and 0≤c≤0.05); LiₐMn_{2-b}X_{b}O_{4-c}D_{c} (0.90≤a≤1.8, 0≤b≤0.5, and O≤c≤0.05); LiₐNi_{1-b-c}Co_{b}X_{c}O_{2-α}D_{α} (0.90≤a≤1.8, 0≤b≤0.5, 0≤c≤0.5, and 0<α<2); LiₐNi_{1-b-c}Mn_{b}X_{c}O_{2-α}D_{α} (0.90≤a≤1.8, 0≤b≤0.5, 0≤c≤0.5, and 0<α<2); LiₐNi_{b}Co_{c}L¹_{d}GₑO₂ (0.90≤a≤1.8, 0≤b≤0.9, 0≤c≤0.5, O≤d≤0.5, and 0≤e≤0.1); LiₐNiG_{b}O₂ (0.90≤a≤1.8 and 0.001≤b≤0.1); LiₐCoG_{b}O₂ (0.90≤a≤1.8 and 0.001≤b≤0.1); LiₐMn_{1-b}G_{b}O₂ (0.90≤a≤1.8 and 0.001≤b≤0.1); LiₐMn₂G_{b}O₄ (0.90≤a≤1.8 and 0.001≤b≤0.1); LiₐMn_{1-b}G_{b}PO₄ (0.90≤a≤1.8 and 0≤g≤0.5); Li_{(3-f)}Fe₂(PO₄)₃ (0≤f≤2); or LiₐFePO₄ (0.90≤a≤1.8).

In the above Chemical Formulas, A is or includes at least one of Ni, Co, Mn, or a combination thereof; X is or includes at least one of Al, Ni, Co, Mn, Cr, Fe, Mg, Sr, V, a rare earth element or a combination thereof; D is or includes at least one of O, F, S, P, or a combination thereof; G is or includes at least one of Al, Cr, Mn, Fe, Mg, La, Ce, Sr, V, or a combination thereof; and L¹ is or includes at least one of Mn, Al, or a combination thereof.

The positive electrode active material may be or include, for example, a high nickel-based positive electrode active material having a nickel content that is greater than or equal to about 80 mol %, greater than or equal to about 85 mol %, greater than or equal to about 90 mol %, greater than or equal to about 91 mol %, or greater than or equal to about 94 mol % and less than or equal to about 99 mol % based on 100 mol % of the metal excluding lithium in the lithium transition metal composite oxide. The high-nickel-based positive electrode active material may be capable of realizing high capacity and can be applied to a high-capacity, high-density rechargeable lithium battery.

### Negative Electrode 20

The negative electrode 20 for a rechargeable lithium battery may include a current collector COL2 and a negative electrode active material layer AML2 on the current collector COL2. The negative electrode active material layer AML2 may include a negative electrode active material, and may further include a binder and/or a conductive material (e.g., an electrically conductive material).

For example, the negative electrode active material layer AML2 may include about 90 wt % to about 99 wt % of the negative electrode active material, about 0.5 wt % to about 5 wt % of the binder, and about 0 wt % to about 5 wt % of the conductive material.

The binder may be configured to sufficiently attach the negative electrode active material particles to each other and also to sufficiently attach the negative electrode active material to the current collector COL2. The binder may include a non-aqueous binder, an aqueous binder, a dry binder, or a combination thereof.

The non-aqueous binder may include at least one of polyvinyl chloride, carboxylated polyvinyl chloride, polyvinyl fluoride, an ethylene propylene copolymer, polystyrene, polyurethane, polytetrafluoroethylene, polyvinylidene fluoride, polyethylene, polypropylene, poly amideimide, polyimide, or a combination thereof.

The aqueous binder may include at least one of a styrene-butadiene rubber, a (meth)acrylated styrene-butadiene rubber, a (meth)acrylonitrile-butadiene rubber, (meth)acrylic rubber, a butyl rubber, a fluoro rubber, polyethylene oxide, polyvinylpyrrolidone, polyepichlorohydrine, polyphosphazene, poly(meth)acrylonitrile, an ethylene propylene diene copolymer, polyvinylpyridine, chlorosulfonated polyethylene, latex, a polyester resin, a (meth)acrylic resin, a phenol resin, an epoxy resins, polyvinyl alcohol, and a combination thereof.

When an aqueous binder is used as the negative electrode binder, a cellulose-based compound capable of imparting viscosity may be further included. The cellulose-based compound may include at least one of carboxymethyl cellulose, hydroxypropylmethyl cellulose, methyl cellulose, or an alkali metal salt thereof. The alkali metal may include at least one of Na, K, or Li.

The dry binder may be or include a polymer material that is capable of being fibrous. For example, the dry binder may be or include polytetrafluoroethylene, polyvinylidene fluoride, a polyvinylidene fluoride-hexafluoropropylene copolymer, polyethylene oxide, or a combination thereof.

The conductive material may be used to impart conductivity (e.g., electrical conductivity) to the electrode. Any material that does not cause chemical change (e.g., that does not cause an undesirable chemical change in the rechargeable lithium battery) and that conducts electrons can be used in the battery. Non-limiting examples thereof may include a carbon-based material such as at least one of natural graphite, artificial graphite, carbon black, acetylene black, ketjen black, a carbon fiber, a carbon nanofiber, and a carbon nanotube; a metal-based material including at least one of copper, nickel, aluminum, silver, etc. in a form of a metal powder or a metal fiber; a conductive polymer such as a polyphenylene derivative; or a mixture thereof.

The negative current collector COL2 may include at least one of a copper foil, a nickel foil, a stainless steel foil, a titanium foil, a nickel foam, a copper foam, a polymer substrate coated with a conductive metal, or a combination thereof.

### Negative Electrode Active Material

The negative electrode active material may include a material that reversibly intercalates/deintercalates lithium ions, a lithium metal, a lithium metal alloy, a material capable of doping/dedoping lithium, or a transition metal oxide.

The material that reversibly intercalates/deintercalates lithium ions may include a carbon-based negative electrode active material, such as, for example. crystalline carbon, amorphous carbon or a combination thereof. The crystalline carbon may be graphite such as non-shaped, sheet-shaped, flake-shaped, sphere-shaped, or fiber-shaped natural graphite or artificial graphite. The amorphous carbon may be a soft carbon, a hard carbon, a mesophase pitch carbonization product, calcined coke, and the like.

The lithium metal alloy includes an alloy of lithium and a metal including at least one of Na, K, Rb, Cs, Fr, Be, Mg, Ca, Sr, Si, Sb, Pb, In, Zn, Ba, Ra, Ge, Al, and Sn.

The material capable of doping/dedoping lithium may be or include a Si-based negative electrode active material or a Sn-based negative electrode active material. The Si-based negative electrode active material may include at least one of silicon, a silicon-carbon composite, SiOx (0<x<2), a Si-Q alloy (where Q includes at least one of an alkali metal, an alkaline-earth metal, a Group 13 element, a Group 14 element (excluding Si), a Group 15 element, a Group 16 element, a transition metal, a rare earth element, and a combination thereof). The Sn-based negative electrode active material may include Sn, SnO₂, a Sn-based alloy, or a combination thereof.

The silicon-carbon composite may be or include a composite of silicon and amorphous carbon. According to an example embodiment, the silicon-carbon composite may be in the form of, or include, silicon particles and amorphous carbon coated on the surface of the silicon particles. For example, the silicon-carbon composite may include a secondary particle (core) in which primary silicon particles are assembled, and an amorphous carbon coating layer (shell) on the surface of the secondary particle. The amorphous carbon may also be between the primary silicon particles, and, for example, the primary silicon particles may be coated with the amorphous carbon. The secondary particles may be dispersed in an amorphous carbon matrix.

The silicon-carbon composite may further include crystalline carbon. For example, the silicon-carbon composite may include a core including crystalline carbon, and silicon particles and an amorphous carbon coating layer on a surface of the core.

The Si-based negative electrode active material or the Sn-based negative electrode active material may be combined with a carbon-based negative electrode active material.

### Separator 30

Depending on the type of the rechargeable lithium battery, the separator 30 may be between the positive electrode 10 and the negative electrode 20. The separator 30 may include at least one of polyethylene, polypropylene, polyvinylidene fluoride, or a multilayer film of two or more layers thereof, and a mixed multilayer film such as a polyethylene/polypropylene two-layer separator, polyethylene/polypropylene/polyethylene three-layer separator, polypropylene/polyethylene/polypropylene three-layer separator, and the like.

The separator 30 may include a porous substrate and a coating layer including an organic material, an inorganic material, or a combination thereof on one or both surfaces of the porous substrate.

The porous substrate may be or include a polymer film formed of or including any one polymer polyolefin including at least one of polyethylene and polypropylene, polyester such as polyethylene terephthalate and polybutylene terephthalate, polyacetal, polyamide, polyimide, polycarbonate, polyether ketone, polyarylether ketone, polyether ketone, polyetherimide, polyamideimide, polybenzimidazole, polyethersulfone, polyphenylene oxide, a cyclic olefin copolymer, polyphenylene sulfide, polyethylene naphthalate, a glass fiber, TEFLON, and polytetrafluoroethylene, or a copolymer or mixture of two or more thereof.

The organic material may include a polyvinylidene fluoride-based polymer or a (meth)acrylic polymer.

The inorganic material may include inorganic particles including at least one of Al₂O₃, SiO₂, TiO₂, SnO₂, CeO₂, MgO, NiO, CaO, GaO, ZnO, ZrO₂, Y₂O₃, SrTiO₃, BaTiO₃, Mg(OH)₂, boehmite, and a combination thereof, but is not limited thereto.

The organic material and the inorganic material may be mixed in one coating layer, or a coating layer including an organic material and a coating layer including an inorganic material may be stacked.

### Electrolyte Solution ELL

The electrolyte solution ELL for a rechargeable lithium battery may include a non-aqueous organic solvent and a lithium salt.

The non-aqueous organic solvent may constitute a medium for transmitting ions taking part in the electrochemical reaction of a battery.

The non-aqueous organic solvent may be or include at least one of a carbonate-based, ester-based, ether-based, ketone-based, or alcohol-based solvent, an aprotic solvent, or a combination thereof.

The carbonate-based solvent may include at least one of dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), and the like.

The ester-based solvent may include at least one of methyl acetate, ethyl acetate, n-propyl acetate, dimethyl acetate, methyl propionate, ethyl propionate, decanolide, mevalonolactone, valerolactone, caprolactone, and the like.

The ether-based solvent may include at least one of dibutyl ether, tetraglyme, diglyme, dimethoxyethane, 2-methyltetrahydrofuran, 2,5-dimethyltetrahydrofuran, tetrahydrofuran, and the like. In addition, the ketone-based solvent may include cyclohexanone, and the like. The alcohol-based solvent may include at least one of ethanol, isopropyl alcohol, and the like and the aprotic solvent may include nitriles such as R—CN (wherein R is a C2 to C20 linear, branched, or cyclic hydrocarbon group, a double bond, an aromatic ring, or an ether bond, and the like; amides such as dimethylformamide; dioxolanes such as 1,3-dioxolane, 1,4-dioxolane, and the like; sulfolanes, and the like.

The non-aqueous organic solvents may be used alone or in combination of two or more.

In addition, when using a carbonate-based solvent, a cyclic carbonate and a chain carbonate may be mixed and used, and the cyclic carbonate and the chain carbonate may be mixed in a volume ratio of about 1:1 to about 1:9.

The lithium salt dissolved in the organic solvent supplies lithium ions in a battery, enables a basic operation of a rechargeable lithium battery, and improves transportation of the lithium ions between positive and negative electrodes. Examples of the lithium salt include at least one of LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, Li(FSO₂)₂N (lithium bis(fluorosulfonyl)imide, LiFSI), LiC₄F₉SO₃, LiN(CₓF₂ₓ₊₁SO₂)(C_{y}F_{2y+1}SO₂) (wherein x and y are integers of 1 to 20), lithium trifluoromethane sulfonate, lithium tetrafluoroethanesulfonate, lithium difluoro(oxalato)borate(LiDFOB), lithium difluorobis(oxalato)phosphate (LiDFBOP), and lithium bis(oxalato) borate (LiBOB).

### Rechargeable Lithium Battery

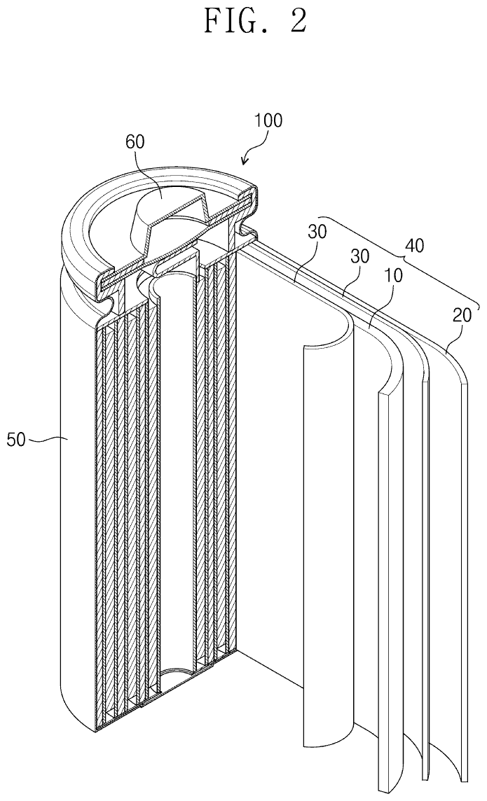

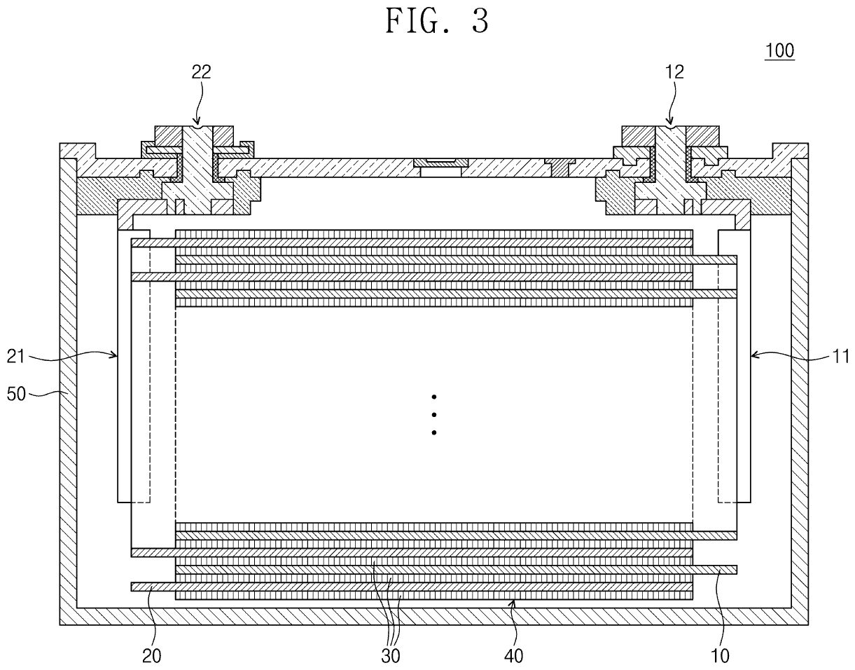

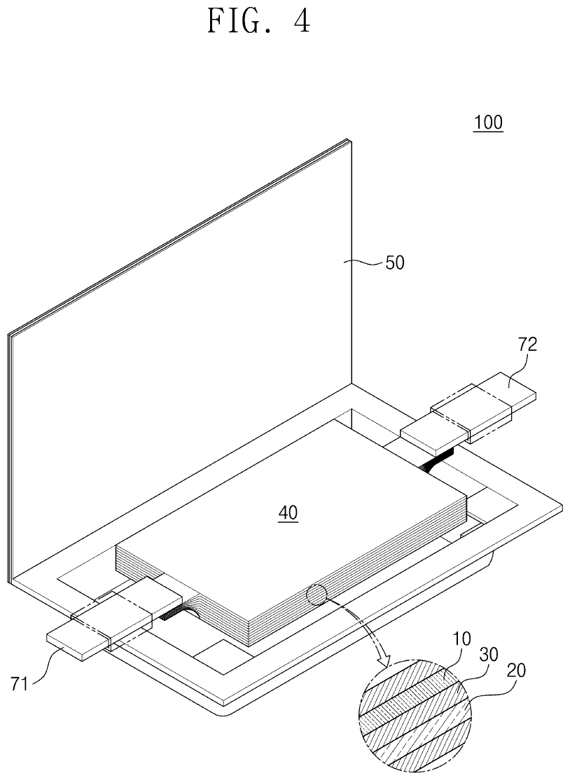

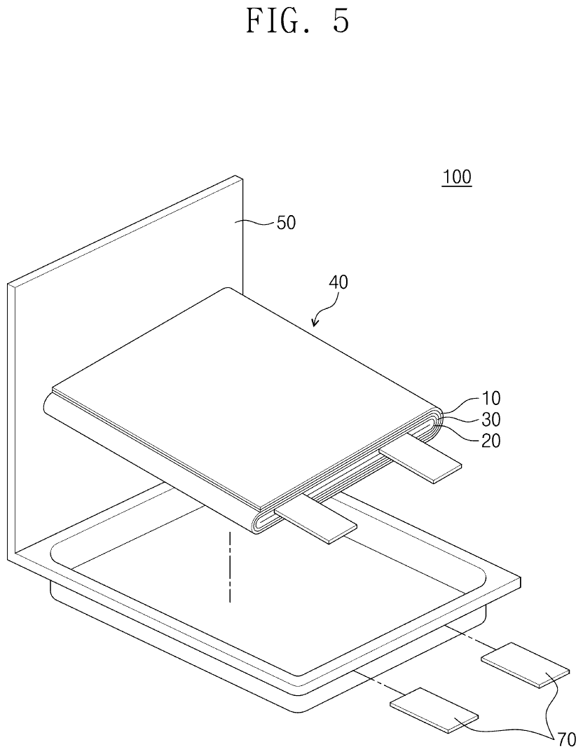

The rechargeable lithium battery may be classified into cylindrical, prismatic, pouch, or coin-type batteries, and the like depending on the shape thereof. FIGS. 2 to 5 are schematic views illustrating a rechargeable lithium battery according to an example embodiment. FIG. 2 shows a cylindrical battery, FIG. 3 shows a prismatic battery, and FIGS. 4 and 5 show pouch-type batteries. Referring to FIGS. 2 to 5, the rechargeable lithium battery 100 may include an electrode assembly 40 including a separator 30 between a positive electrode 10 and a negative electrode 20, and a case 50 in which the electrode assembly 40 is included. The positive electrode 10, the negative electrode 20, and the separator 30 may be impregnated with an electrolyte solution (not shown). The rechargeable lithium battery 100 may include a sealing member 60 sealing the case 50, as shown in FIG. 2. In FIG. 3, the rechargeable lithium battery 100 may include a positive lead tab 11, a positive terminal 12, a negative lead tab 21, and a negative terminal 22. As shown in FIGS. 4 and 5, the rechargeable lithium battery 100 may include an electrode tab 70 illustrated in FIG. 5, which may be, for example, a positive electrode tab 71 and a negative electrode tab 72 illustrated in FIG. 4 and constituting an electrical path for inducing the current formed in the electrode assembly 40 to the outside of the battery 100.

Hereinafter, an electrolyte solution of a rechargeable lithium battery according to example embodiments of the present disclosure will be described in more detail.

The electrolyte solution for a rechargeable lithium battery according to an example embodiment includes a non-aqueous organic solvent, a lithium salt, and an additive.

The additive according to an example embodiment of the present disclosure may be represented by Formula 1 below:

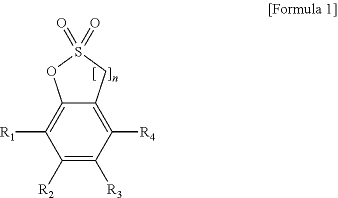

In Formula 1 above,

R₁ to R₄ may each independently be a hydrogen atom, a halogen atom, or a substituted or unsubstituted C1 to C10 alkyl group, and n may be an integer ranging from 1 to 5. A case in which n is 0 may indicate a direct linkage. For example, in the case that n is equal to 0, a cyclic sulfur derivative moiety may be a pentagonal ring.

R₁ to R₄ in Formula 1 above may be a hydrogen element. For example, Formula 1 above may be represented by Formula 1-1 or Formula 1-2 below.

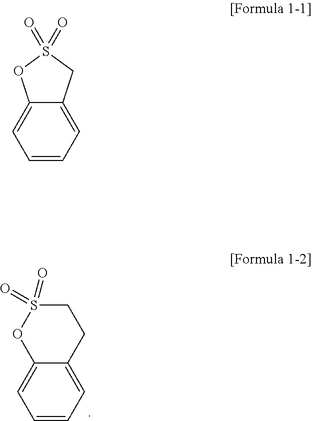 [SMILES: O=S1(=O)Cc2ccccc2O1 ; O=S1(=O)CCc2ccccc2O1]

In an example embodiment, the electrolyte additive for a rechargeable lithium battery according to the present disclosure may be or include a compound represented by Formula 1-1 below:

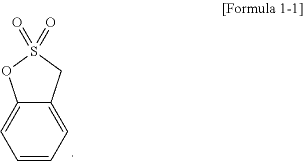 [SMILES: O=S1(=O)Cc2ccccc2O1]

The additive according to an example embodiment of the present disclosure may be or include an aromatic ring compound having a two-ring structure in which a ring containing a sulfonic acid (—SO₃—) group and a benzene ring are fused.

The additive according to an example embodiment of the present disclosure has a sulfonic acid (—SO₃—) functional group, and may thus form a stable film on a surface of a positive electrode, thereby reducing or suppressing decomposition of a positive electrode active material. Accordingly, gas generation resulting from the decomposition of a positive electrode active material may be reduced or suppressed.

The benzene ring included in the additive according to an example embodiment of the present disclosure may reduce or suppress the decomposition of a positive electrode active material by forming a film on a surface of a positive electrode through oxidation and formation of a cathode-electrolyte interphase (CEI). Accordingly, gas generation resulting from the decomposition of a positive electrode active material may be reduced or suppressed.

The additive according to an example embodiment of the present disclosure may have a more stable and stronger bonding force through the fusion of the aromatic benzene ring and carbon atoms placed next to an oxygen atom of the sulfone ring. These structural characteristics reduce or suppress the elution of transition metals, and may be effective in reducing or suppressing positive electrode deterioration.

Accordingly, through the two-ring structure, the additive according to an example embodiment of the present disclosure may more effectively contribute to the stability and cycle life characteristics of lithium batteries at high voltage.

The additive may be included in an amount of about 0.01 wt % to about 10 wt % with respect to a total amount of the electrolyte solution. For example, the amount of the additive may be in a range of about 0.05 wt % to about 3 wt % with respect to the total amount of the electrolyte solution. When the content of the additive is less than the above range, a film may not be fully formed on lithium-based positive and negative electrodes, and when the content of the additive is greater than the above range, batteries may have reduced capacity and lifetime due to increased resistance of the positive and negative electrodes.

The electrolyte solution according to an example embodiment may be prepared by a method through a mixing process in which a lithium salt is dissolved in a non-aqueous organic solvent, and the additive is added. The process of mixing the electrolyte solution may be known in the field of electrolyte solution preparation, and will be appropriately selected and used by a person skilled in the art.

The non-aqueous organic solvent may include at least one of ethyl methyl carbonate (EMC), ethylene carbonate (EC), dimethyl carbonate (DMC), propylene carbonate (PC), propylpropionate (PP), diethyl carbonate (DEC), dipropyl carbonate (DPC), methylpropyl carbonate (MPC), ethylpropyl carbonate (EPC), methylethyl carbonate (MEC), and butylene carbonate (BC).

For example, the non-aqueous organic solvent may be or include a mixed solvent of ethyl methyl carbonate (EMC), ethylene carbonate (EC), and dimethyl carbonate (DMC).

For example, the ethyl methyl carbonate (EMC) may be included in an amount of about 30 vol % to about 50 vol % with respect to the total amount of the non-aqueous organic solvent. The ethylene carbonate (EC) may be included in an amount of about 10 vol % to about 30 vol % with respect to the total amount of the non-aqueous organic solvent. The dimethyl carbonate (DMC) may be included in an amount of about 30 vol % to about 50 vol % with respect to the total amount of the non-aqueous organic solvent.

The lithium salt may be or include at least one of LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, Li(FSO₂)₂N, (lithium bis(fluorosulfonyl)imide (LiFSI)), and LiC₄F₉SO₃. According to an example embodiment, LiPF₆ may be used as the lithium salt.

The lithium salt may have a concentration of about 0.1 M to about 2.0 M. For example, the lithium salt may have a concentration of about 0.5 M or greater or about 1.0 M or greater. The lithium salt may have a concentration of about 2.0 M or less, about 1.7 M or less, or about 1.5 M or less. In the present disclosure, when the lithium salt has a concentration of about 0.1 M to about 2.0 M, conductivity and viscosity the electrolyte solution may be appropriately maintained.

In another example embodiment of the present disclosure, a rechargeable lithium battery which includes a positive electrode including a positive electrode active material, a negative electrode including a negative electrode active material, and an electrolyte solution (where a non-aqueous organic solvent, a lithium salt, and the above-described additive represented by Formula 1 described above are included) may be provided.

The positive electrode active material of the rechargeable lithium battery may include at least one of cobalt-free nickel-manganese-based oxide, lithium nickel-based oxide, lithium cobalt-based oxide, lithium manganese-based oxide, a lithium iron phosphate-based compound, and a combination thereof may be used. Specifically, for example, and cobalt-free nickel-manganese-based oxide.

In the rechargeable lithium battery using the electrolyte solution according to the present disclosure, the negative electrode active material includes at least one of a carbon-based negative electrode active material, a Si-based negative electrode active material, a Sn-based negative electrode active material, or a combination thereof.

The silicon-based negative electrode active material may include a core containing Si-based particles and a coating layer containing amorphous carbon. The Si-based particles may include at least one of silicon particles, a silicon-carbon composite, SiOₓ (0<x ≤2), or a Si alloy.

The rechargeable lithium battery may operate at a high voltage of about 4.4 V or greater.

The rechargeable lithium battery may be used in, e.g., automobiles, mobile phones, and/or various types of electric devices, as non-limiting examples.

Hereinafter, Examples and Comparative Examples of the present disclosure will be described. However, the following Examples are presented only as an example embodiment of the present disclosure, and the present disclosure is not limited to the Examples below.

### EXAMPLES AND COMPARATIVE EXAMPLES

### Synthesis Example 1

0.1 mmol of 2-hydroxybenzyl alcohol and 0.125 mmol of sodium bisulfite are added dropwise to a Schlenk flask containing 100 ml of H₂O, and then reflux-stirred for 24 hours. Thereafter, an excess amount of phosphoryl chloride is added to the compound obtained by filtering the precipitate, and the mixture is subjected to a reaction without a solvent (neat) at 125° C. for 1 hour. Subsequently, the compound represented by Formula 1-1 below is obtained through purification using a Soxhlet extractor:

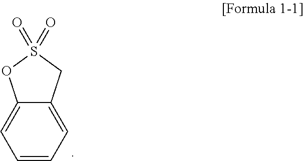 [SMILES: O=S1(=O)Cc2ccccc2O1]

*1H NMR (400 MHz, CDCl₃): δ 4.50 (s, 2H), 7.10 (d, JZ8.2 Hz, 1H) 7.20 (dd, JZ8.2, 7.6 Hz, 1H), 7.33 (d, JZ7.4 Hz, 1H), 7.40 (dd, JZ7.6, 7.4 Hz, 1H).

### Example 1

### (1) Preparation of Electrolyte Solution

1.15 M LiPF₆ was dissolved in a non-aqueous organic solvent in which ethylene carbonate (EC), ethyl methyl carbonate (EMC), and dimethyl carbonate (DMC) were mixed in a volume ratio of 20:40:40, and 0.5 wt % of an additive was added to prepare an electrolyte solution.

The additive represented by Formula 1-1 prepared in Synthesis Example 1 was used.

### (2) Preparation of Rechargeable Lithium Battery

97 wt % of NMX (LiNiMnO) as a positive electrode active material, 0.5 wt % of artificial graphite powder, 0.8 wt % of carbon black (Ketjenblack), 0.2 wt % of acrylonitrile rubber, and 1.5 wt % of polyvinylidene fluoride (PVdF) as a conductive material were mixed and added to N-methyl-2-pyrrolidone, and then the mixture was stirred for 30 minutes using a mechanical stirrer to prepare a positive electrode active material slurry. Using a doctor blade, the slurry was applied onto a 20 μm-thick aluminum current collector to be 60 μm thick, dried in a hot air dryer at 100° C. for 0.5 hours, dried again under vacuum at 120° C. for 4 hours, and then roll pressed to prepare a positive electrode.

98 wt % of negative electrode active material in which artificial graphite and a Si composite were mixed at a weight ratio of 93:7, 1 wt % of styrene-butadiene rubber (SBR), and 1 wt % of carboxymethyl cellulose (CMC) were mixed, and then added to distilled water, and stirred for 60 minutes using a mechanical stirrer to prepare a negative electrode active material slurry. Using a doctor blade, the slurry was applied onto a 10 μm-thick copper current collector to be 60 μm thick, dried in a hot air dryer at 100° C. for 0.5 hours, dried again under vacuum at 120° C. for 4 hours, and then roll pressed to prepare a negative electrode.

An electrode assembly was prepared by assembling the positive electrode, the negative electrode, and a 10 μm-thick polyethylene separator, and the electrolyte solution was injected to prepare a rechargeable lithium battery.

### Example 2

An electrolyte solution and a rechargeable lithium battery were prepared in the same manner as in Example 1, with a difference that 0.1 wt % of the additive was used.

### Example 3

An electrolyte solution and a rechargeable lithium battery were prepared in the same manner as in Example 1, with a difference that 1.0 wt % of the additive was used.

### Example 4

An electrolyte solution and a rechargeable lithium battery were prepared in the same manner as in Example 1, with a difference that 3.0 wt % of the additive was used.

### Example 5

An electrolyte solution and a rechargeable lithium battery were prepared in the same manner as in Example 1, with a difference that the additive represented by Formula 1-2 below was used.

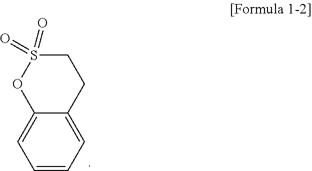 [SMILES: O=S1(=O)CCc2ccccc2O1]

### Comparative Example 1

An electrolyte solution and a rechargeable lithium battery were prepared in the same manner as in Example 1, with a difference that the additive represented by Formula 1-1 was not used during the preparation of the electrolyte solution.

### Comparative Example 2

An electrolyte solution and a rechargeable lithium battery were prepared in the same manner as in Example 1, with a difference that the additive represented by Formula 1-3 below was used.

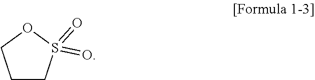

### Evaluation Example

The rechargeable lithium batteries were evaluated in the following manner.

### Evaluation 1: Evaluation of High Temperature Storage Characteristics (DC-IR Change Rate)

ΔV/ΔI (change in voltage/change in current) values for initial DC-IR with respect to the rechargeable lithium batteries prepared according to Examples and Comparative Examples were measured, the maximum energy state inside the batteries was subsequently set to a fully charged state (SOC 100%) and stored at high temperature (60° C.) for 30 days in this condition, and DC-JR was measured and the DC-JR increase rate (%) was calculated according to Equation 1 below, and the results are shown in Table 1 below.

$$DC - IR increase rate = ( DC - IR after 30 days / initial DC - IR ) \qquad Equation 1$$

TABLE 1

| | | DC-IR after | |
|---|---|---|---|
| | | storage at 60° C. | |
| | Initial DC-IR | for 30 days | DC-IR |
| Item | mohm | mohm | % |
| Comparative | 42.3 | 53.0 | 125.4 |
| Example 1 | | | |
| Comparative | 39.8 | 51.1 | 128.5 |
| Example 2 | | | |
| Example 1 | 37.5 | 44.4 | 118.3 |
| Example 2 | 38.1 | 48.4 | 127.1 |
| Example 3 | 38.4 | 47.7 | 124.3 |
| Example 4 | 38.9 | 49.2 | 126.5 |
| Example 5 | 37.9 | 45.3 | 119.4 |

### Evaluation 2: Evaluation of High-Temperature Gas Generation Characteristics

High-temperature gas generation characteristics were evaluated for the rechargeable lithium batteries according to Examples and Comparative Examples. For this purpose, the rechargeable lithium batteries according to Examples and Comparative Examples were charged to about 4.25 V at about 45° C. and then left standing at about 60° C. for about 7 days.

In order to determine the gas reduction effect, the initial gas generation amount of cell batteries and the gas generation amount of cell batteries after being left for 7 days were each measured, and the results are shown in Table 2 below.

TABLE 2

Gas generation amount (mL) at high
temperature (60° C.) storage

| Item | Day 1 | Day 7 |
|---|---|---|
| Comparative | 0.033 | 0.080 |
| Example 1 | | |
| Comparative | 0.030 | 0.067 |
| Example 2 | | |
| Example 1 | 0.018 | 0.034 |
| Example 2 | 0.024 | 0.048 |
| Example 3 | 0.027 | 0.057 |
| Example 4 | 0.028 | 0.062 |
| Example 5 | 0.021 | 0.040 |

### Evaluation 3: Evaluation of Room Temperature and High Temperature Charge/Discharge Cycle Characteristics

The rechargeable lithium batteries prepared in Examples and Comparative Examples were subjected to 0.33 C charge (CC/CV, 4.45 V cut-off)/1.0 C discharge (CC, 3.0 V cut-off) at room temperature (25° C.) and high temperature (45° C.) for 200 cycles, and then discharge capacity was measured and capacity retention was calculated, and the results are shown in Table 3 below. The capacity retention was calculated according to Equation 2 below.

$$Capacity retention ( \% ) = ( discharge capacity after 200 cycles / initial discharge capacity ) * 100 \qquad Equation 2$$

TABLE 3

| | Capacity retention (%) at | Capacity retention (%) at |
|---|---|---|
| Item | room temperature (25° C.) | high temperature (45° C.) |
| Comparative | 89.1 | 81.1 |
| Example 1 | | |
| Comparative | 89.9 | 83.5 |
| Example 2 | | |
| Example 1 | 92.3 | 89.3 |
| Example 2 | 91.5 | 88.7 |
| Example 3 | 90.9 | 87.9 |
| Example 4 | 90.1 | 87.4 |
| Example 5 | 92.1 | 89.1 |

### Comprehensive Evaluation

Referring to Table 1, when electrolyte solutions containing the additive according to an example embodiment of the present disclosure (Examples 1 to 5) were used exhibited improved storage properties at high temperature (60° C.) compared to the electrolyte solution with no additive (Comparative Example 1).

Referring to Table 2, the rechargeable lithium batteries prepared according to Comparative Examples were found to generate a large amount of gas when stored at high temperature (60° C.) compared to the rechargeable lithium batteries prepared according to Examples 1 to 5. Accordingly, the rechargeable lithium battery using the additive represented by specifically Formula 1 according to the present disclosure may effectively reduce or suppress the generation of gas at high temperature (60° C.).

Comparative Example 2, in which a cyclic compound containing a sulfonic acid (—SO₃—) group alone was used as an additive, was found to have a large amount of gas when stored at a relatively high temperature (60° C.) compared to Examples.

Referring to Table 3, an electrolyte solution containing the additive according to an example embodiment of the present disclosure has improved cycle characteristics and life efficiency of the batteries at room temperature, and high temperature storage conditions, compared to Comparative Examples.

An electrolyte solution for a rechargeable lithium battery according to an example embodiment both stabilizes electrodes and reduces or suppresses an increase in resistance, and may thus produce the effect of improving lifetime characteristics and stability at high temperatures.

Although example embodiments of the present disclosure have been described above, the scope of the present disclosure is not limited to the example embodiments. Various modifications of the example embodiments may be made without departing from the spirit and scope of the disclosure as defined by the appended claims, and the modifications are included in the scope of the present disclosure.

What is claimed is:

1. An electrolyte solution for a rechargeable lithium battery, the electrolyte solution comprising:
a non-aqueous organic solvent;
a lithium salt; and
an additive represented by Formula 1 below: 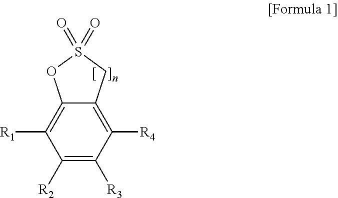
wherein in Formula 1 above,
R₁ to R₄ are each independently a hydrogen atom, a halogen atom, or a substituted or unsubstituted C₁ to C₁₀ alkyl group, and
n is an integer ranging from 1 to 5.

2. The electrolyte solution for a rechargeable lithium battery of claim 1, wherein the additive is represented by one of Formula 1-1 and Formula 1-2 below:

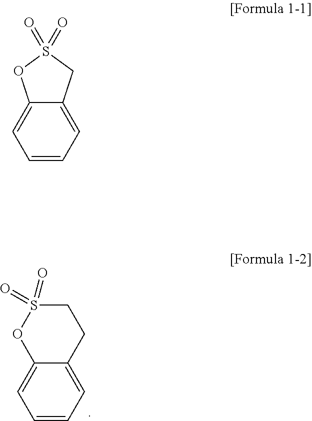 [SMILES: O=S1(=O)Cc2ccccc2O1 ; O=S1(=O)CCc2ccccc2O1]

3. The electrolyte solution for a rechargeable lithium battery of claim 1, wherein the additive amounts to about 0.05 wt % to about 3 wt % with respect to a total weight of the electrolyte solution.

4. The electrolyte solution for a rechargeable lithium battery of claim 1, wherein the non-aqueous organic solvent comprises at least one of ethyl methyl carbonate (EMC), ethylene carbonate (EC), dimethyl carbonate (DMC), propylene carbonate (PC), propylpropionate (PP), diethyl carbonate (DEC), dipropyl carbonate (DPC), methylpropyl carbonate (MPC), ethylpropyl carbonate (EPC), methylethyl carbonate (MEC), and butylene carbonate (BC).

5. The electrolyte solution for a rechargeable lithium battery of claim 1, wherein the lithium salt comprises at least one of LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, Li(FSO₂)₂N, (lithium bis(fluorosulfonyl)imide (LiFSI)), and LiC₄F₉SO₃.

6. The electrolyte solution for a rechargeable lithium battery of claim 1, wherein the lithium salt has a concentration of about 0.1 M to about 2.0 M.

7. A rechargeable lithium battery comprising:
a positive electrode including a positive electrode active material;
a negative electrode including a negative electrode active material; and
the electrolyte solution according to claim 1.

8. The rechargeable lithium battery of claim 7, wherein the positive electrode active material comprises at least one of cobalt-free nickel-manganese-based oxide, lithium nickel-based oxide, lithium cobalt-based oxide, lithium manganese-based oxide, and a lithium iron phosphate-based compound.

9. The rechargeable lithium battery of claim 7, wherein the negative electrode active material comprises at least one of a carbon-based negative electrode active material, a Si-based negative electrode active material, and a Sn-based negative electrode active material.

10. The rechargeable lithium battery of claim 9, wherein the Si-based negative electrode active material comprises a silicon-carbon composite.

11. The rechargeable lithium battery of claim 7, wherein the rechargeable lithium battery is configured to operate at a high voltage of about 4.4 V or greater.

12. A compound represented by Formula 1-1 below: 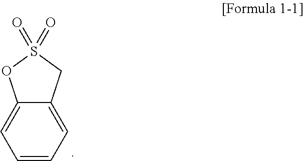 [SMILES: O=S1(=O)Cc2ccccc2O1]
